(19) United States
(12) Patent Application Publication
(10) Pub. No.: US 2025/0281025 A1
(43) Pub. Date: Sep. 11, 2025

(54) Probe Tip for in Vivo Multi-Photon Measurement

(71) Applicant: Trustees of Tufts College, Boston, MA (US)

(72) Inventors: Irene GEORGAKOUDI, Winchester, MA (US); Adriana HERNANDEZ-SANCHEZ, Medford, MA (US)

(73) Assignee: Trustees of Tufts College, Boston, MA (US)

(21) Appl. No.: 18/854,391

(22) PCT Filed: Apr. 5, 2023
(86) PCT No.: PCT/US2023/017611

§ 371 Date: Oct. 4, 2024

Related U.S. Application Data
(60) Provisional application No. 63/329,157, filed on Apr. 8, 2022.

(51) Int. Cl. A61B 1/00; A61B 1/06; A61B 1/12; A61B 90/00

(52) U.S. Cl. CPC A61B 1/00096; A61B 1/00089; A61B 1/00101; A61B 1/00105; A61B 1/00128; A61B 1/0669; A61B 1/127; A61B 90/39; A61B 2090/3933

(57) ABSTRACT

Probe tip structures are disclosed that can replaccably couple to an imaging probe to provide a sterile interface between an imaging probe and the tissue undergoing analysis. The probe and associated tip apparatus can then be used for noninvasive or minimally invasive in vivo microscopy to visualize the surface or lining of internal organs and/or tissue. The tip apparatus can be attached to the probe before (or during the procedure) and then removed and discarded, such that the probe, itself, remains relatively free of contamination and more readily sterilizable for reuse. Alternatively, the tip apparatus can be sterilizable and reusable. The tip structures are particularly useful in conjunction with multi-photon imaging probes.

### RELATED APPLICATIONS

This application claims priority to U.S. Provisional Application No. 63/329,157 filed on Apr. 8, 2022, entitled “Probe Tip for in Vivo Multi-Photon Measurement,” which is incorporated herein by reference in its entirety.

### FEDERALLY SPONSORED RESEARCH OR DEVELOPMENT

This invention was made with Government support under Grant Nos. R21-EB023498 and R03-CA235053 awarded by National Institutes of Health. The Government has certain rights in the invention.

### FIELD

The technical field of the invention is in vivo assessment of biological structures with minimally invasive optical probe instruments.

### BACKGROUND

Endoscopic visualization of tissue, e.g., with an endoscope, laparoscope, colonoscope, or the like, is becoming an important tool in detecting and/or staging internal tissue diseases, such as cancers of the cervix, uterus, ovaries, colon, peritoneum, stomach, esophagus, etc. Recent advances in imaging, particularly in multi-photon imaging, have demonstrated that it is possible to distinguish between healthy tissue and cancerous infiltrations by non-invasive or minimally invasive techniques. See, for example, Pouli et al. Two-photon images reveal unique texture features for label-free identification of ovarian cancer peritoneal metastasis,” Biomedical Optics Express 10: 9/1 pp.4479-4489 (2019).

Multiphoton-photon microscopy is a fluorescence imaging technique in which fluorophores within biological structures are excited by simultaneous (or near simultaneous) absorption of two or more photons. The wavelength of the excitation photons, typically in the near-infrared (NIR) range, is longer than the fluorescence emission wavelength, typically in the visible spectrum. Multi-photon excitation microscopy, using NIR light, can reduce scattering in the tissue and suppress background noise, leading to better imaging and the ability to more readily distinguish between healthy and diseased tissue.

However, multi-photon endoscopic probes are complex optical instruments. Consequently, they are expensive and must be re-usable. Reducing contact between the instrument, itself, and the biological structures that are undergoing examination would reduce the amount of sterilization necessary for probe reuse.

### SUMMARY

This disclosure relates generally to a probe system including a tip apparatus that can reduce contact between the probe, itself, and the biological structures that are undergoing examination, thereby reducing the amount of sterilization necessary for probe reuse. In some embodiments, probe tip apparatuses are disclosed that can replaceably couple to an imaging probe to provide a sterile interface between an imaging probe and the tissue undergoing analysis. The probe and associated tip apparatus can then be used for minimally invasive in vivo microscopy to visualize the surface or lining of internal organs and/or tissue. The tip apparatus can be attached to the probe before (or during the procedure) and then removed and discarded, such that the probe, itself, remains relatively free of contamination and more readily sterilizable for reuse. Alternatively, the tip apparatus can be sterilizable and reusable. A tip apparatus according to the present teachings is particularly useful in conjunction with multi-photon imaging probes.

In one embodiment, the tip apparatus can include a tubular body having an internal hollow space therethrough such that images of a target site, e.g., multi-photon excited fluorescence images, can be captured by the probe. In certain embodiments, the tip apparatus can also be a conduit for a fiberscope to guide accurate probe positioning. The proximal end of the tip apparatus (the end that connects to the probe) can include a coupler that facilitates quick and simple connection. The coupler can comprise, for example, a threaded, or snap-seal or twist-and-seal linkage between the tip apparatus and the probe.

The distal end of the tip apparatus can comprise a window, which can be brought into contact (or into proximity) with the target biological structure to facilitate both excitation of the target and imaging.

The window at the distal end of the tip apparatus preferably is an optically clear, high mechanical strength, plate optic or lens. In certain embodiments, the window comprises glass or sapphire. Sapphire can be particularly advantageous due to its surface hardness and resistance to abrasion and scratching as well as its wide optical transmission band from ultraviolet to mid-infrared wavelengths. In some embodiments, the window includes an antifogging coating to prevent fogging during imaging.

In certain embodiments, the distal end of the tip apparatus can also include a rim such that the window is recessed. The rim at the distal end of the tip structure can enable good contact with the tissue and inhibit, and preferably block, entry of any background illumination into the probe, which can be important for acquisition of two photon images.

In some embodiments, the distal end can also include an index-matching fluid to reduce reflections and/or aberrations during imaging within the tip apparatus. For example, the index-matching fluid can be water, silicone, acrylic, or any other optically transmissive fluid that serves to reduce or eliminate reflection losses associated with a glass-air interface. The fluid can be a liquid, oil or a gel.

In certain embodiments where the imaging probe has an objective lens or the like at its distal end, the tip is configured to cover or encased the probe's distal end with a fluid reservoir that fills the space between the probe's distal lens and the tip's window, thereby eliminating or reducing any air gaps that might otherwise be present when the tip is coupled to the imaging probe.

In certain embodiments, the tip apparatus can comprise a tubular body or sheath formed from a material that can be sterilized, such as stainless steel, a recessed window coupled to the tubular body and formed from an optically clear material (e.g. glass or sapphire). The tip can be attached to a multiphoton probe via threads or a clip mechanism to ensure the distance between the inner surface of the window and the distal end of the multi-photon probe is controlled. The window can be connected to the tubular body or sheath via any material that is water-tight and biocompatible. Water or refractive index matching material is present between the tip and the multi-photon probe during imaging to minimize reflections and aberrations.

The tip apparatus can be coupled to the imaging probe beforehand such that the tip apparatus is already connected to the imaging probe upon insertion. For example, the tip can be screwed onto a smaller diameter distal end of the imaging probe and then inserted through a cannula as part of a probe assembly.

The tip assemblies disclosed herein can also address the need to have a clear reference point of contact between the probe and the surface of the tissue in order to acquire depth resolved images with high accuracy. The disposition of a refractive index matching fluid between the probe and the tip also provides for a means to acquire images with minimal aberrations.

The presence of a lip/rim at the end of the tip apparatus also enables good contact with the tissue and ideally blocks most or all background illumination, which can likewise be important for acquisition of multiphoton images.

In some preferred embodiments, the tip apparatus can be sterilizable and reusable. For example, the tip apparatus (or at least a portion of it, such as the tube body) can be constructed from a material that can withstand sterilization, such as 316 stainless steel.

### BRIEF DESCRIPTION OF THE DRAWINGS

A brief description of each drawing is provided to more sufficiently understand drawings used in the detailed description of the present teachings.

FIGS. 1A and 1B schematically depict a probe and a tip apparatus in accordance with an exemplary embodiment;

FIG. 2A and 2B schematically depict a tip apparatus in accordance with an exemplary embodiment;

FIG. 3A schematically depicts a proximal end of a tip apparatus in accordance with an exemplary embodiment;

FIG. 3B schematically depicts an end of a tip apparatus in accordance with an exemplary embodiment;

FIG. 4 schematically depicts a probe and a tip apparatus in accordance with an exemplary embodiment;

FIG. 5 schematically depicts a probe and a tip apparatus in accordance with an exemplary embodiment;

FIG. 6 schematically depicts a tip apparatus including guide tubes in accordance with an exemplary embodiment;

FIGS. 7A and 7B schematically depicts a tip apparatus including guide tubes in accordance with an exemplary embodiment; and

FIG. 8 schematically depicts a tip apparatus including an adhesive in accordance with an exemplary embodiment.

It should be understood that the above-referenced drawings are not necessarily to scale, presenting a somewhat simplified representation of various preferred features illustrative of the basic principles of the present teachings. The specific design features of the present teachings, including, for example, specific dimensions, orientations, locations, and shapes, will be determined in part by the particular intended application and use environment.

### DETAILED DESCRIPTION

Advantages and features of the present teachings and a method of achieving the same will become apparent with reference to the accompanying drawings and embodiments described below in detail. However, the present teachings are not limited to the embodiments described herein and may be embodied in variations and modifications. The embodiments are provided merely to allow one of ordinary skill in the art to understand the scope of the present teachings, which will be defined by the scope of the claims. Accordingly, in some embodiments, well-known operations of a process, well-known structures, and well-known technologies will not be described in detail to avoid obscure understanding of the present teachings. Throughout the specification, same reference numerals refer to same elements.

The terminology used herein is for the purpose of describing particular embodiments only and is not intended to be limiting of the present teachings. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

“Endoscope” is an instrument that is typically used for in vivo diagnostic or therapeutic purposes, having a flexible or rigid tubular optical fiber to obtain optical images (still or video) of internal organs or tissues. The term “endoscopy” refers to the diagnostic or therapeutic procedure using an endoscope. Types of endoscopy include arthroscopy, bronchoscopy, colonoscopy, cystoscopy, enteroscopy, colposcopy, hysteroscopy, laparoscopy, laryngoscopy, mediastinoscopy, sigmoidoscopy, thoracoscopy (also known as pleuroscopy), gastroscopy (or esophagogastroduodenoscopy), ureteroscopy, and the like.

“Laparoscope” is a type of an endoscope for performing laparoscopy, and more specifically refers to a fiber-optic instrument inserted through an incision in the abdominal wall to examine visually the interior of the peritoneal cavity in the abdominal or pelvic area.

The term “imaging probe” or “probe,” as used herein, can refer to an endoscope, a laparoscope, a multi-photon imaging endoscope, or any other similar optical diagnosis apparatus. Herein, the description of the “imaging probe” or “probe” is provided primarily with regards to medical applications. However, the present teachings are not limited thereto, and the imaging system can be similarly applied to endoscopes or imaging probes for industrial applications. An aspect of the present teachings provides a multi-photon imaging system for in vivo measurement. The imaging system can provide more effective and economical means of sterilization for reuse, and can also provide a structure that can exclude ambient light that may interfere the measurement.

Hereinbelow, the multi-photon imaging system according to the present teachings will be described with reference to the appended drawings.

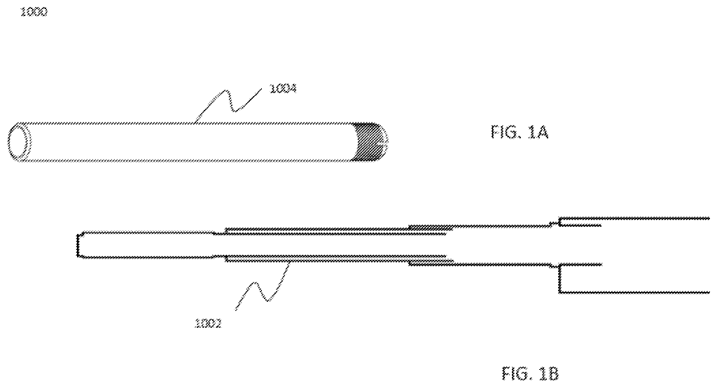

FIGS. 1A and 1B schematically show a system 1000 according to an embodiment of the present teachings for multiphoton imaging, e.g., multiphoton imaging of tissue. The system 1000 for multiphoton imaging includes an imaging probe 1002 and a tip apparatus 1004 that can be detachably and replaceably coupled to the imaging probe 1002, e.g., in a manner discussed further below. In this embodiment, the imaging probe 1002 is a multi-photon, e.g., a two-photon, imaging probe. However, the present teachings are not limited to multi-photon probes. In fact, the imaging probe 1002 can be any type of non-linear optical imaging probe.

The system 1000 may include a coupler 1005. The coupler 1005 can be configured to attach the tip apparatus 1004 to the imaging probe 1002 before use. In certain embodiments, coupling can be achieved using a threaded coupler, i.e., by screwing on via threads formed both on an outer surface of the imaging probe 1002 and on an inner surface of the coupler 1005. In certain embodiments, a snap-seal coupler or a twist-and-seal coupler (e.g., a lure-lock type) can be used. For snap-seal coupling, one of the outer surface of the imaging probe 1002 or the inner surface of the coupler 1005 can include a coupling groove, and the other can include a coupling protrusion. Additionally, or alternatively, an O-ring or a gasket can be included between the outer surface of the imaging probe 1002 and the inner surface of the coupler 1005. The present teachings are not limited thereto, however, and any other means for gas/liquid sealing can be used between the tip apparatus 1004 and the imaging probe 1002.

In certain embodiments, a body of the tip apparatus and the coupler 1005 can be integrally formed. Alternatively, the body and the coupler 1005 can be separately formed and then mechanically joined. The body and the coupler 1005 are preferably made of a biocompatible metal or polymer. Since, in certain embodiments, the tip apparatus can be sterilized and reused, the body 100 and the coupler 1005 can be constructed using a material that can withstand repeated sterilization. By way of example, a medical grade stainless steel (e.g., 316 stainless steel), titanium, nickel-titanium alloy (nitinol), gold, platinum, or silver can be used.

The tip apparatus 1004 includes a window 1008. The window 1008 can be configured to transmit the excitation light (e.g., NIR laser) from the imaging probe 1002 as well as the light emitted from the fluorophores of the biological structures. In certain embodiments, the window 1008 can be made of glass (e.g., break resistant glass). In certain embodiments, the window 1008 can be made of sapphire or silica. Glass can be more suitable where the imaging probe 1002 is a polarization-sensitive probe whereas sapphire can be used for a non-polarization-sensitive probe. Sapphire exhibits improved surface hardness and resistance to abrasion and/or scratching as well as wide optical transmission band from ultraviolet to mid-infrared wavelengths. In some embodiments, an antireflective coating or antifogging coating may be applied to the window 1008.

As noted above, the rim at the distal end of the tip apparatus can allow controlling the distance between the inside surface of the window 1008 and the distal tip of the imaging probe 1002. However, the cavity between the window and the tip of the imaging probe 1002 can create a glass-air interface. Due to the difference of the refractive indices for air and the material of window 1008, light may suffer losses, and/or the image may the distorted due to reflection, and refraction. In order to minimize or reduce reflection losses and/or aberrations, the cavity between the tip of the imaging probe 1002 and the window 1008 of the tip apparatus 1004 can be filled with an index-matching fluid 1011. In other words, the cavity between the distal end of the imaging probe 1002 and the window 1008 of the tip apparatus 1004 can be used as a reservoir for the index-matching fluid 1011.

The index-matching fluid 1011 can be in the form of a liquid, oil, or a gel, and can include water, silicone, acrylic, or any other optically transmissive fluid. By way of example, when the window 1008 is made of sapphire, the refractive index of which is about 1.7-1.8, antimony tribromide (SbBr₃) salt dissolved in liquid diiodomethane (CH₂I₂) can be used. For glass, the refractive index of which is about 1.4-1.5, ultrasound gel and silicone-based fluids/gels can be used. Preferably, the index-matching fluid 1011 can include biocompatible substances.

In some embodiments, a rim 1009 that is disposed at the distal end of the body 100 can surround the window 1008 and protrude distally outward therefrom along the axial direction of the imaging probe 1002. By way of example, the protrusion distance of the rim 1009 can be about 0.5 mm to about 5 mm. When the tip apparatus 1004 is pressed onto the imaging target site or an in vivo site (e.g., tissue or organ) to be analyzed, the rim 1009 can provide a seal between the target site and the window 1008 of the tip apparatus 1004, such that the tip apparatus 1004 is in good contact with the target site and any background illumination that can interfere with the measurement is substantially (and preferably completely) blocked from entering the imaging probe 1002. Maintaining an optical seal from the ambient light can be important for acquiring reliable multi-photon images.

Another aspect of the present teachings provides a method of performing multi-photon imaging (e.g., microscopy) in vivo, for example, during a laparoscopy. In many embodiments, a tip apparatus according to the present teachings and a method according to the present teachings for performing multi-photon imaging can provide a means to better exclude ambient light for improved image qualities, and also a more convenient and reliable means for sterilization of the probe system.

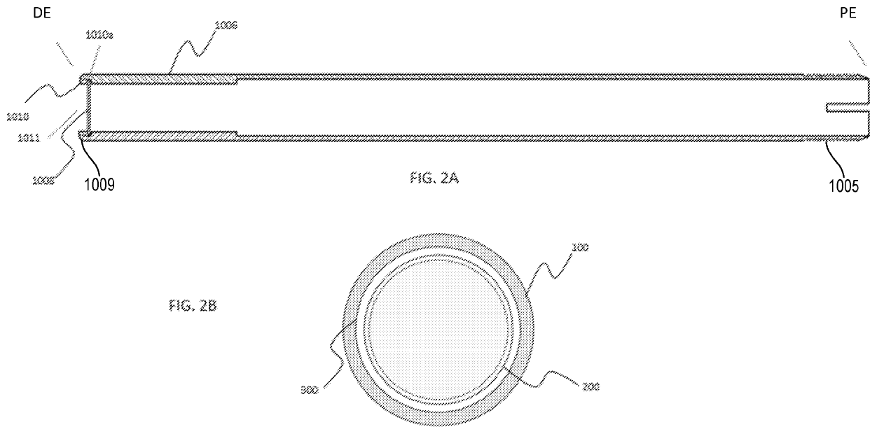

With reference to FIGS. 2A and 2B, the tip apparatus 1004 can include a body 1006, a window 1008, and a window coupler 1010. In this embodiment, the body 1006 extends from a proximal end (PE) to a distal end (DE) and has a tubular shape providing a hollow lumen that is configured to receive a portion of the distal end of the imaging probe 1002. In this embodiment, the window coupler 1010 includes a shoulder 1010a on which the window 1008 can be seated and held in place via a retaining ring 1008a. In this embodiment, the window is recessed relative to the distal end of the probe tip such that a recess cavity 1011 is formed between the distal end of the tip apparatus and the window.

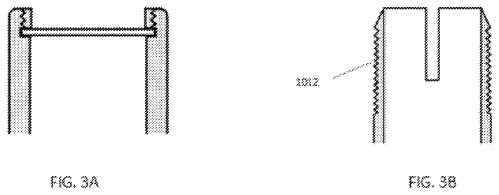

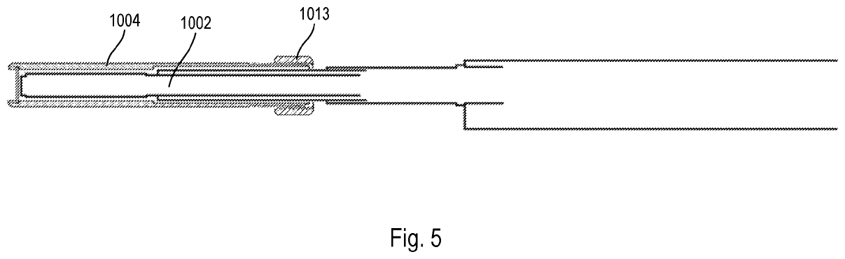

FIGS. 3A and 3B schematically depict longitudinal cross-sections of the distal and the proximal ends of the tip apparatus 1004, respectively. The proximal end of the tip apparatus can include a mechanism for detachably and replaceably coupling the tip apparatus to a distal portion of the imaging probe. By way of example, a plurality of threads 1012 provided on an outer surface of the distal end of the tip apparatus can engage with a plurality of mating threads provided on an inner surface portion of the distal end of the imaging probe. In another embodiment (FIG. 5), the mechanism for coupling the tip apparatus to the distal portion of the imaging probe can include a nut 1013 that can screw onto the outside of the sheath to provide secure attachment of the tip apparatus to the imaging probe.

In certain embodiments, the tip apparatus can be formed of a material that can be readily sterilized. By way of example, the tip apparatus can be formed of stainless steel.

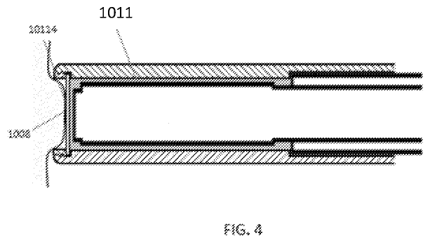

FIG. 4 schematically shows that the cavity formed between the distal end of the tip apparatus and the window creates a tissue-window interface 1014. Further, FIG. 4 shows that in this embodiment, once the tip apparatus is coupled to the imaging probe, a cavity 1015 is formed between the tip apparatus and the imaging probe.

Due to the difference of the refractive indices of air and the material from which window 1008 is formed (e.g., glass), light reflected and/or scattered back from a tissue portion received into the cavity 1011 may suffer losses and/or an image of the tissue portion formed via detection of such light may exhibit distortions, e.g., due reflection, refraction and/or diffraction.

In some embodiments, the cavity 1015 formed between the imaging probe and the tip apparatus can be filled with an index-matching fluid 1011 in order to minimize, and preferably eliminate, such reflection losses and/or aberrations. Some examples of suitable index-matching fluids can include, without limitation, water ultrasound gel, and refractive index matching oil.

Further, the recess of the optically clear window 1008 from the tip of the imaging probe can advantageously minimize the leakage of light from surrounding environment, including potential illumination sources into the imaging probe.

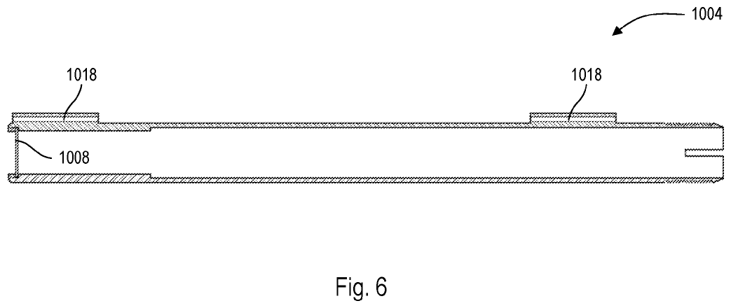

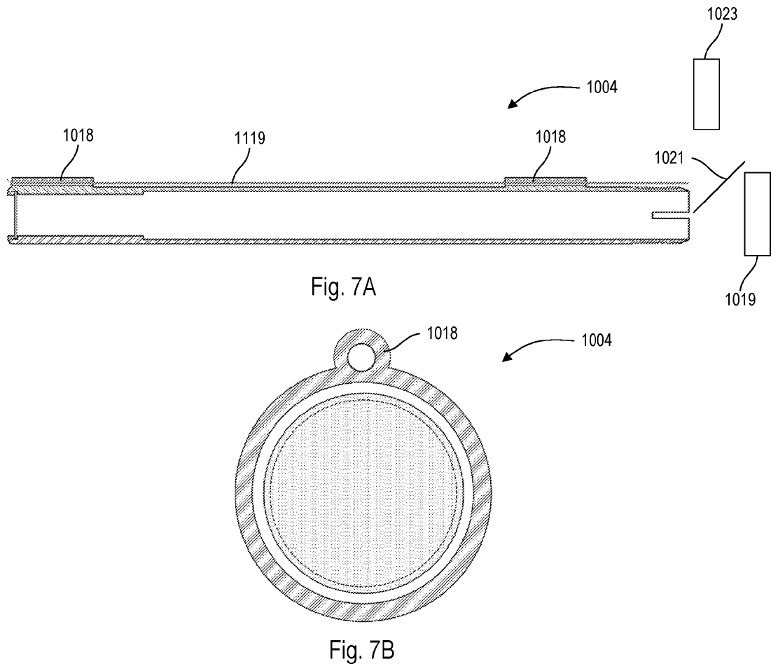

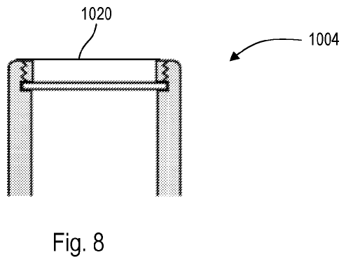

With reference to FIGS. 6, 7A and 7B in some embodiments, the tip apparatus 1004 includes guide tubes 1018 for a fiberscope. As depicted in FIG. 7A, one or more channels 1018 can be provided in the body of the tip apparatus to receive one or more optical fiber(s) 1119.

As depicted in FIG. 7A, the system 1000 may include a light source 1019, a beam splitter 1021, and a detector 1023. Light that is emitted by the light source 1019 is coupled into a proximal end of the optical fiber(s) via passage through the beam splitter 1021, where the optical fiber(s) transmit the received light to its distal end through which the light exits the optical fiber(s) to illuminate the tissue, and the light reflected/scattered by the illuminated tissue (or at least a portion thereof) is captured by the optical fiber(s) and is transmitted to the proximal end of the optical fiber through which the returning light exits the optical fiber(s) and is reflected by the beam splitter 1021 to be received by the detector 1023.

The tip apparatus 1004 also includes a biocompatible adhesive 1020 (e.g., a biocompatible adhesive tape) located at the distal end of the tip apparatus 1020. The adhesive 1020 stabilizes the imaging probe 1002 during imaging.

The tip apparatus 1004 further includes marking elements. When the marking elements contact a biological structure, the marking elements may provide a visual indicator on the biological structure. For example, the marking elements may inject visible ink into the biological structure. In some embodiments, the adhesive 1020 includes the marking elements.

Hereinabove, although the present teachings are described by specific matters such as concrete components, and the like, the embodiments and the drawings are provided merely for assisting in the entire understanding of the present teachings. Therefore, the present teachings are not limited to the embodiments described herein. Various modifications and changes can be made by a person of ordinary skill in the art to which the present teachings pertain. The spirit of the present teachings should not be limited to the above-described embodiments, and the following claims as well as all technical spirits modified equally or equivalently to the claims should be interpreted to fall within the scope and spirit of the present teachings.

1. A replaceable tip apparatus for an imaging probe, the tip apparatus comprising:
a tubular body having an internal hollow space therein;
a proximal coupler configured to attach the tip apparatus to the imaging probe; and
a distal window to transmit light from an in vivo site to optics within the imaging probe.

2. The apparatus of claim 1, wherein the imaging probe is a multi-photon imaging probe, having a distal lens for image capture, and
wherein the tip apparatus upon coupling to the imaging probe forms a reservoir for receiving an index-matching fluid between the distal lens of the probe and the window of the tip apparatus.

3. The apparatus of claim 1, wherein the apparatus further comprises a distal rim such that the window is recessed for an imaging target site and background light is blocked from interference with imaging.

4. The apparatus of claim 1, wherein the proximal coupler is threaded coupler, or snap-seal or twist-and-seal coupler.

5. The apparatus of claim 1, wherein the window comprises glass.

6. The apparatus of claim 1, wherein the window comprises sapphire.

7. A method of performing multi-photon microscopy in vivo, the method comprising:
coupling a replaceable tip apparatus to a multi-photon imaging probe;
inserting the imaging probe toward an imaging target site;
pressing a distal window of the tip apparatus onto the imaging target site, thereby blocking background light; and
obtaining an axial scan of the imaging target site using the imaging probe.

8. The method of claim 9, wherein the method further comprises:
prior to coupling the tip apparatus to the imaging probe, filling an index-matching fluid within the tip apparatus.

9. The method of claim 9, further comprising:
removing the tip apparatus from the imaging probe; and
sterilizing the tip apparatus.

10. The tip apparatus of claim 1, wherein the window includes an antireflective or antifogging coating.

11. The tip apparats of claim 1, further comprising a heating element configured to thermally stabilize the tip apparatus and defog the window.

12. The tip apparatus of claim 1, further comprising a guide element configured to position the imaging probe.

13. The tip apparatus of claim 18, wherein the guide elements are further configured to illuminate tissue and detect reflected light.

14. The tip apparatus of claim 1, further comprising a biocompatible adhesive at a proximal end of the tip apparatus and wherein the adhesive stabilizes the imaging probe when contacting a biological structure.

15. The tip apparatus of claim 1, further comprising marking elements configured to provide a visual indicator on a biological structure.

16. The tip apparatus of claim 1, wherein the marking elements are configured to inject visible ink into a biological structure.
